(19) United States
(12) Patent Application Publication
(10) Pub. No.: US 2023/0238087 A1
(43) Pub. Date: Jul. 27, 2023

(54) SYSTEM AND METHOD FOR OPTIMIZING TRIAL DESIGN FOR CLINICAL TRIALS

(71) Applicant: Innoplexus AG, Eschborn (DE)

(72) Inventors: Nitish Jain, Pune (IN); Vipul Vinod Patni, Shrirampur (IN); Nishant Singhania, Jamshedpur (IN); Vismay Bansal, Mount Abu (IN); Bheru Mali, Bhilwara (IN)

(73) Assignee: Innoplexus AG, Eschborn (DE)

(21) Appl. No.: 17/575,021

(22) Filed: Jan. 13, 2022

(51) Int. Cl. G16H 10/20; G16H 50/20; G16H 50/70

(52) U.S. Cl. CPC G16H 10/20; G16H 50/20; G16H 50/70

(57) ABSTRACT

A system and method for optimizing trial design for clinical trials. The system includes a computer system and a processor communicably coupled to a memory. The processor processes and structures raw trial data to a format suitable for input to train a machine learning model. The processor further identifies plurality of independent features of the raw trial data and screens actionable features. Further, the processor computes cut off range values for each of the actionable features and form a plurality of sub-groups of patients. The processor simulates patient response of each of the plurality of sub-groups of patients and identifies a sub-group of patients based upon population percentage and delta response that shows optimal clinical trial results in the simulated patient response.

### TECHNICAL FIELD

The present disclosure relates generally to clinical trials; and more specifically, to system and method for optimizing trial designs for clinical trials.

### BACKGROUND

In the recent past, clinical trials are required for getting a new drug approved by a regulatory agency like the FDA (Federal Drug Administration). Additionally, the effect of a new therapeutic or diagnostic test on humans needs to be proven by following a clearly defined test procedure that is described in detail in a clinical trial protocol. Moreover, after approval of the protocol by an ethics committee, a trial sponsor recruits clinical sites and patients for the trial. Furthermore, the necessary procedures are initiated, and clinical data is generated, stored, and validated according to the protocol description. Notably, it takes between 10 and 15 years and costs between $1.5 and $2.0 billion to bring a new drug to market. Additionally, despite many advancements in science and technology, the number of drugs approved have been declining steadily since the past 70 years according to “Eroom’s Law” by Scannell et al. (2012). Moreover, about half of this time and money is dedicated to conducting clinical trials but they still have a high rate of failure. Furthermore, choosing the optimum trial design parameters along with the right population who can benefit the most from the intervention, hence showing clear impacts of the same is of utmost importance to the success of a trial.

Notably, both clinical studies and follow-on formal clinical trials are traditionally time-consuming, costly, and often incomplete. Additionally, many of these trials end unsuccessfully, not only because of operational difficulties, but also due to more fundamental issues of selecting the wrong hypotheses or inappropriate patient cohorts. Moreover, whether a clinical trial is conducted through a contract research organization (CRO) or by recruiting investigators, access to patient cohorts remains a bottleneck in the clinical trial process. Currently, cohorts are selected either through open participation, by using media for recruitment, or by relying on clinical investigators, who are often selected from academic medical centres and hospitals to identify appropriate cohorts from their respective patient bases.

Typically, conventional methods for identifying patients for clinical studies manually are effective in some instances. However, there are several problems associated with conventional patient identifying methods. For example, the selection process is expensive and time-consuming. Additionally, patient identifying and selection costs more and consumes more time than any other aspect of clinical trials. In fact, more than 80% of clinical trials suffer from delays. Moreover, patient identification accounts for 41% of the time spent on clinical research. Furthermore, the delays associated with patient identification inevitably delays the introduction of new drugs and therapies to the public.

Therefore, in light of the foregoing discussion, there exists a need to overcome the aforementioned drawbacks associated with a trial design in a clinical trial.

### SUMMARY

The present disclosure seeks to provide a system for optimizing trial design for clinical trials. The present disclosure also seeks to provide a method for optimizing trial design for clinical trials. An aim of the present disclosure is to provide a solution that overcomes at least partially the problems encountered in prior art.

In one aspect, the present disclosure provides a system for optimizing trial design for clinical trials, wherein the system includes a computer system comprising a processor communicably coupled to a memory, the processor operable to:
process and structure raw trial data to a format suitable for input to train a machine learning model, wherein the raw trial data is patient data;
identify plurality of independent features of the raw trial data, wherein the identification of the plurality of independent features is performed using the trained machine learning model;
screen actionable features from the plurality of independent features using the trained machine learning model, wherein actionable features show opposite impact between treatment arm patients and control arm patient of the clinical trial;
compute cut off range values for each of the actionable features, and form a plurality of sub-groups of patients using different combinations of cut-off range values, wherein the cut off range values define an upper limit and a lower limit for values of the actionable features;
simulate patient response of each of the plurality of sub-groups of patients; and
identify a sub-group of patients from the plurality of sub-groups, based upon population percentage and delta response obtained from the simulations, that shows optimal clinical trial results in the simulated patient response.

In another aspect, the present disclosure provides a method for optimizing trial design for clinical trials, wherein the method comprises:
processing and structuring raw trial data to a format suitable for input to train a machine learning model using a processor, wherein the raw trial data is patient data;
identifying plurality of independent features of the raw trial data, wherein the identification of the plurality of independent features is performed using the trained machine learning model;
screening actionable features from the plurality of independent features using the trained machine learning model, wherein actionable features show opposite impact between treatment arm patients and control arm patient of the clinical trial;
computing cut off range values for each of the actionable features, and form a plurality of sub-groups of patients using different combinations of cut-off range values, wherein the cut off range values define an upper limit and a lower limit for values of the actionable features;
simulating patient response of each of the plurality of sub-groups of patients; and
identifying a sub-group of patients from the plurality of sub-groups, based upon population percentage and delta response obtained from the simulations, that shows optimal clinical trial results in the simulated patient response.

Embodiments of the present disclosure substantially eliminate or at least partially address the aforementioned problems in the prior art, and increases the chance of a successful clinical trial by selecting a right group of patients.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is schematic illustration of a system for optimizing trial design for clinical trials, in accordance with an embodiment of the present disclosure;

FIG. 2 is a plot between percentage population and delta response, in accordance with an exemplary implementation of the present disclosure; and

FIG. 3 is a flowchart depicting steps of a method for optimizing trial design for clinical trials, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, the present disclosure provides a system for optimizing trial design for clinical trials, wherein the system includes a computer system comprising a processor communicably coupled to a memory, the processor operable to:
process and structure raw trial data to a format suitable for input to train a machine learning model, wherein the raw trial data is patient data;
identify plurality of independent features of the raw trial data, wherein the identification of the plurality of independent features is performed using the trained machine learning model;
screen actionable features from the plurality of independent features using the trained machine learning model, wherein actionable features show opposite impact between treatment arm patients and control arm patient of the clinical trial;
compute cut off range values for each of the actionable features, and form a plurality of sub-groups of patients using different combinations of cut-off range values, wherein the cut off range values define an upper limit and a lower limit for values of the actionable features;
simulate patient response of each of the plurality of sub-groups of patients; and
identify a sub-group of patients from the plurality of sub-groups, based upon population percentage and delta response obtained from the simulations, that shows optimal clinical trial results in the simulated patient response.

In another aspect, the present disclosure provides a method for optimizing trial design for clinical trials, wherein the method comprises:
processing and structuring raw trial data to a format suitable for input to train a machine learning model using a processor, wherein the raw trial data is patient data;
identifying plurality of independent features of the raw trial data, wherein the identification of the plurality of independent features is performed using the trained machine learning model;
screening actionable features from the plurality of independent features using the trained machine learning model, wherein actionable features show opposite impact between treatment arm patients and control arm patient of the clinical trial;
computing cut off range values for each of the actionable features, and form a plurality of sub-groups of patients using different combinations of cut-off range values, wherein the cut off range values define an upper limit and a lower limit for values of the actionable features;
simulating patient response of each of the plurality of sub-groups of patients; and
identifying a sub-group of patients from the plurality of sub-groups, based upon population percentage and delta response obtained from the simulations, that shows optimal clinical trial results in the simulated patient response.

The system and method of the present disclosure aims to provide optimization of trial design in a clinical trial. Notably, the present disclosure reduces the time and cost in selecting the right group of patients for the clinical trial. Consequently, the system eliminates the delay in the overall process of drug discovery. Furthermore, the present disclosure increases the chance of a successful clinical trial by selecting the right group of patients.

Pursuant to embodiments of the present disclosure, the system and the method provided herein are for optimizing trial design for clinical trials. Herein, “clinical trial” refers to research studies performed in people that are aimed at evaluating a medical, surgical, or behavioral intervention. Additionally, clinical trials are the primary way that researchers find out if a new treatment, like a new drug or diet or medical device (for example, a pacemaker) is safe and effective in people. Moreover, often a clinical trial is used to learn if a new treatment is more effective and/or has less harmful side effects than the standard treatment. Furthermore, clinical trials are conducted using a process that may be divided into categories or phases. Typically, clinical trial process can extend over a period of time ranging from months to years. Notably, every clinical trial requires retrieving, analyzing, and managing the collaboratively obtained clinical trial data from various clinical trial organizations collected during the clinical trial process before an investigational new drug (IND) can be submitted to the FDA.

The system includes a computer system comprising a processor communicably coupled to a memory. Herein, a “computer system” relates to at least one computing unit comprising a central storage system, processing units and various peripheral devices. Optionally, the computer system relates to an arrangement of interconnected computing units, wherein each computing unit in the computer system operates independently and may communicate with other external devices and other computing units in the computer system.

Throughout the present disclosure, the term “processor” used herein relates to a computational element that is operable to respond to and process instructions that carry out the method. Optionally, the processor includes, but is not limited to, a microprocessor, a microcontroller, a complex instruction set computing (CISC) microprocessor, a reduced instruction set (RISC) microprocessor, a very long instruction word (VLIW) microprocessor, or any other type of processing circuit. Furthermore, the term “processor” may refer to one or more individual processors, processing devices and various elements associated with a processing device that may be shared by other processing devices.

The processor is operable to process and structure raw trial data to a format suitable for input to train a machine learning model, wherein the raw trial data is patient data. Herein “raw trial data” refers to unprocessed patient data for a clinical trial that is in its original form, in contrast to derived data. Additionally, raw trial data may not be part of the documentation accompanying an application to a regulatory authority but must be kept in records. Moreover, raw trial data may include patient medical charts, hospital records, X-rays, attending physician’s notes, and so forth. Herein, “machine learning model” refers to the output that is saved after running a machine learning algorithm on training data and represents the rules, numbers, and any other algorithm-specific data structures required to make predictions. Notably, raw trial data requires processing and structuring in a format that is a valid input to the machine learning model. Additionally, the raw trial data is inserted to the machine learning model after processing and structuring it using the processor. Additionally, the processed and structured raw trial data acts as the training data for the machine learning model.

Optionally, the machine learning model is XGBoost regressor, and wherein the XGBoost regressor is trained using grid search. Herein, “XGBoost regressor” or extreme gradient boosting is an open-source library that provides an efficient and effective implementation of the gradient boosting algorithm. Herein, gradient boosting refers to a class of ensemble machine learning algorithms that can be used for classification or regression predictive modeling problems. Additionally, ensembles are constructed from decision tree models. Moreover, trees are added one at a time to the ensemble and fit to correct the prediction errors made by prior models. Notably, this is a type of ensemble machine learning model referred to as boosting. Furthermore, models are fit using any arbitrary differentiable loss function and gradient descent optimization algorithm. Consequently, this gives the technique its name, “gradient boosting,” as the loss gradient is minimized as the model is fit, much like a neural network. Herein, the XGBoost regressor is trained using grid search for tuning the Hyperparameters of the said model. Herein, “hyperparameters” refers to a parameter whose value is used to control the learning process of the machine learning model. By contrast, the values of other parameters are derived via training. Additionally, hyperparameter is a characteristic of a model that is external to the model and whose value cannot be estimated from data. Moreover, the value of the hyperparameter has to be set before the learning process begins. Herein, “grid search” refers to a process that searches exhaustively through a manually specified subset of the hyperparameter space of the targeted algorithm. Furthermore, grid-search is used to find the optimal hyperparameters of a model which results in the most accurate predictions.

The processor is operable to identify plurality of independent features of the raw trial data, wherein the identification of the plurality of independent features is performed using the trained machine learning model. Notably, after processing the data, the next step is identification of the important independent factors that primarily affect outcome of the clinical trial. Optionally, the plurality of independent features comprises at least one of: genetic features, baseline indexes, vital signs, underlying conditions, medical history, and demographics such as age, gender, height, weight, BMI, nationality, race. Furthermore, the machine learning model is run separately for treatment arm patients and control arm patients. Herein, “treatment arm” refers to a group or subgroup of participants in a clinical trial that receives a specific intervention, study drug dose, according to the study protocol. Herein, “control arm” refers to a group or subgroup of participants that do not receive the new medication, device or treatment that is under study, to provide a comparison to see how the innovation compares against no treatment. Additionally, members of the control group may receive a placebo, an inactive treatment such as a pill that makes the group think they are receiving the new treatment.

Optionally, the plurality of independent features have missing values that are imputed using a plurality of imputation techniques, wherein the plurality of imputation techniques employs statistical extrapolation. Herein, “imputation” refers to an assumed value given to an item when the actual value is not known or available. Additionally, imputed values are a logical or implicit value for an item or time set, wherein a true value is yet to be ascertained. Notably, the imputation techniques are used to determine the values of the missing independent features, if any. Moreover, the imputation techniques used are mean, median, mode and so forth. Furthermore, the imputation techniques are implemented based on the features, for example, mean for continuous, mode for categorical, and so forth.

Optionally, the XGBoost regressor identifies the independent features that do not impact efficacy of treatment used in the clinical trial.

The processor is operable to screen actionable features from the plurality of independent features using the trained machine learning model, wherein actionable features show opposite impact between treatment arm patients and control arm patient of the clinical trial. Notably, the actionable features chosen by the processor helps to compare the impact of the new drug between treatment arm patients and control arm patient of the clinical trial. Herein, actionable features refer to the independent features that can be controlled and using which an action relating to selection of patients in the clinical trial can be taken.

Optionally, opposite impact between treatment arm patients and control arm patients is measured as improvement in the treatment arm patients and decrease in efficacy in the control arm patients. Notably, opposite impact between the treatment arm that receives the new drug and control arm that doesn’t receive the new drug means an improvement in the treatment arm patients and decrease in efficacy in the control arm patients. Additionally, the decrease in efficacy in the control arm patients clearly indicates that the new drug tested on the treatment arm patients is working.

The processor is operable to compute cut off range values for each of the actionable features and form a plurality of sub-groups of patients using different combinations of cut-off range values, wherein the cut off range values define an upper limit and a lower limit for values of the actionable features. Notably, the processor applies cut off ranges at all levels of a particular independent factor and results in separation of respective population segments. Additionally, the raw trial data gets segregated into a plurality of sub-groups. Moreover, the number of sub-groups depends on the different combinations of cut-off range values.

The processor is operable to simulate patient response of each of the plurality of sub-groups of patients. Notably, the processor performs a simulation with the independent factors selected by the machine learning model and applies a combination of cutoffs at all levels of these independent factors. Additionally, a delta response is determined for patients meeting the cutoff criteria by simulating the range of important independent variables.

The processor is operable to identify a sub-group of patients from the plurality of sub-groups, based upon population percentage and delta response obtained from the simulations, that shows optimal clinical trial results in the simulated patient response. Notably, each subgroup obtained by the cut-off range values contains patients from the treatment arm as well as from the control arm. Additionally, the average difference in improvement between the two arms is compared statistically and respective p-value is calculated. Moreover, the population percentage after filtering out patients and their delta change in endpoint score is noted. Herein, endpoint score is calculated by subtracting control arm average from the treatment arm average. Furthermore, the sub-group with the best impact of the intervention in the treatment arm compared to the control arm is selected. Herein, the population percentage refers to percentage of patients with respect to total number of patients in each of the plurality of sub-groups.

Optionally, the simulation data, percentage population and delta response are plotted, and the best point is chosen as the point with good delta response and high percentage population. Consequently, this leads to a tradeoff between reducing target population versus proving efficacy. Furthermore, a population sub-group is identified that shows significantly better improvement in the treatment arm compared to the control arm with a population percentage greater than 50 percent to easily meet the recruitment needs.

In an exemplary implementation, the simulation results for different combinations of four independent features for the plurality of sub-groups may be tabulated as follows:

TABLE 1

| Baseline Score 1 | Baseline Score 2 | Time since disease commencement (Years) | Age (Years) | Overall Count | Delta Test Score Change (Treatment -Control) | Population Percentage |
|---|---|---|---|---|---|---|
| - | - | - | - | 145.00 | 0.02 | 100% |
| - | >= x₂₁ | - | - | 81.00 | 1.00 | 56% |
| - | - | >= X₃₁ | - | 75.00 | 0.74 | 52% |
| >= X₁₂ | - | - | - | 74.00 | 1.78 | 51% |
| - | - | - | <= X₄₁ | 72.00 | 1.06 | 50% |
| >= X₁₃ | - | >= X₃₁ | - | 45.00 | 1.56 | 31% |
| >= X₁₁ | - | - | <= X₄₂ | 42.00 | 2.69 | 29% |
| - | >= X₂₂ | >= X₃₂ | - | 36.00 | 0.37 | 25% |
| - | - | >= X₃₂ | <= X₄₃ | 35.00 | 1.76 | 24% |
| >= X₁₄ | >= X₂₃ | - | - | 32.00 | 5.11 | 22% |
| - | >= X₂₄ | - | <= X₄₁ | 31.00 | 1.89 | 21% |
| >= X₁₁ | - | >= X₃₃ | <= X₄₁ | 27.00 | 2.58 | 19% |
| >= X₁₁ | >= X₂₅ | >= X₃₄ | - | 17.00 | 3.98 | 12% |
| >= X₁₁ | >= X₂₁ | - | <= X₄₄ | 14.00 | 4.64 | 10% |
| - | >= X₂₄ | >= X₃₅ | <= X₄₁ | 12.00 | 2.25 | 8% |
| >= X₁₅ | >= X₂₃ | >= X₃₅ | <= X₄₅ | 9.00 | 4.50 | 6% |

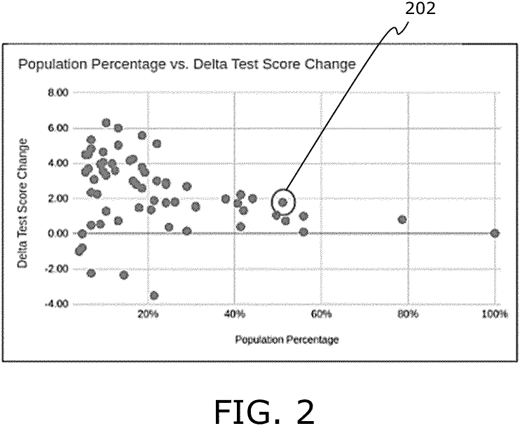

The corresponding plot between percentage population and delta response is illustrated in FIG. 2.

The present disclosure also relates to the method as described above. Various embodiments and variants disclosed above apply mutatis mutandis to the method.

Optionally, the method comprises imputing the missing values of the plurality of independent features using a plurality of imputation techniques, wherein the plurality of imputation techniques employ statistical extrapolation.

Optionally, the method comprises training XGBoost regressor using grid search, wherein the machine learning model is XGBoost regressor.

Optionally, the method comprises identifying the independent features that do not impact efficacy of treatment used in the clinical trial using the XGBoost regressor.

Optionally, the method comprises the plurality of independent features to be at least one of: genetic features, baseline indexes, vital signs, underlying conditions, medical history, and demographics such as age, gender, height, weight, BMI, nationality, race.

Optionally, the method comprises measuring opposite impact between treatment arm patients and control arm patients as improvement in the treatment arm patients and decrease in efficacy in the control arm patients.

### DETAILED DESCRIPTION OF THE DRAWINGS

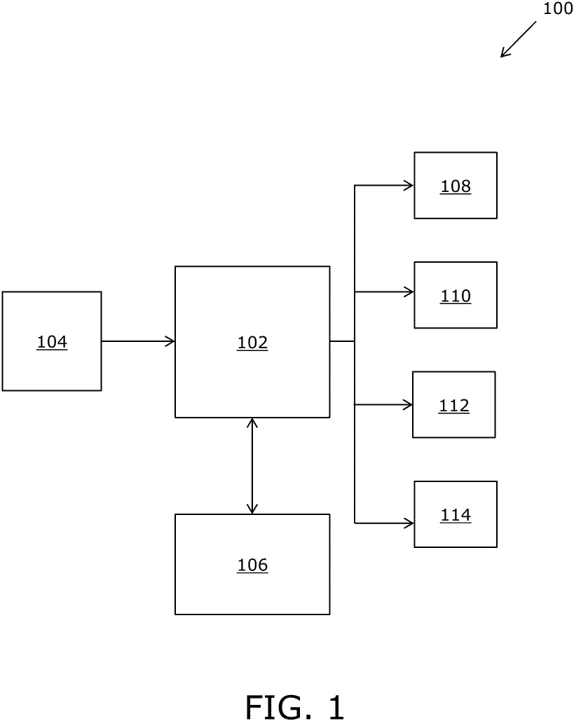

Referring to FIG. 1, illustrated is a schematic illustration of a system 100 for optimizing trial design for clinical trials, in accordance with an embodiment of the present disclosure. The system comprises a processor 102 communicably coupled to a memory (not shown). The processor 102 is operable to process and structure raw trial data 104 to a format suitable for input to train a machine learning model 106. The processor 102 identifies plurality of independent features of the raw trial data 104 and screens actionable features from the plurality of independent features using the trained machine learning model. The actionable features show opposite impact between treatment arm patients and control arm patient of the clinical trial. The processor further computes cut off range values for each of the actionable features, and form a plurality of sub-groups, such as sub-groups 108, 110, 112, 114 of patients using different combinations of cut-off range values. The processor further simulates patient response of each of the plurality of sub-groups, such as sub-groups 108, 110, 112, 114 of patients and identifies a sub-group of patients based upon population percentage and delta response that shows optimal clinical trial results in the simulated patient response.

Referring to FIG. 2, illustrated is a plot between percentage population and delta response, in accordance with an exemplary implementation of the present disclosure. The plot provides a distribution of a plurality of sub-groups with respect to their corresponding percentage population and delta response as provided in Table 1. Notably, the sub-group represented by the point 202 may be selected as it shows significantly better improvement in the Treatment arm compared to the Control arm with a population percentage >50% that easily meets recruitment needs.

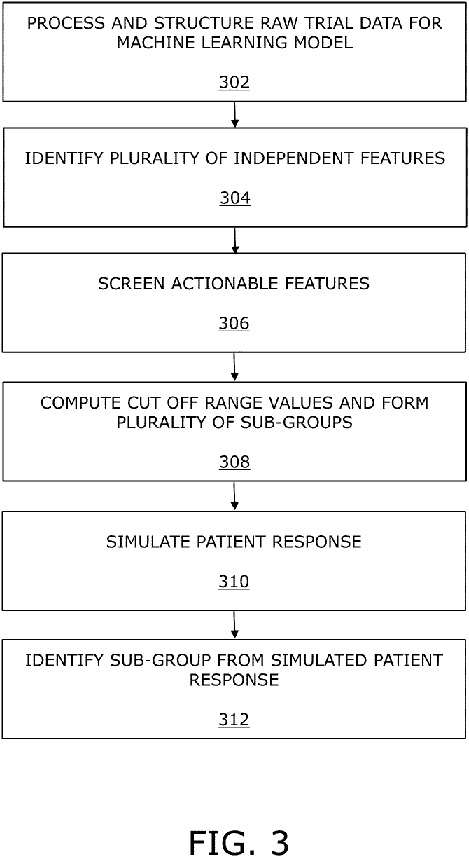

Referring to FIG. 3, illustrated is a flowchart depicting steps of a method for optimizing trial design for clinical trials, in accordance with an embodiment of the present disclosure. At step 302, raw trial data is processed and structured to a format suitable for input to train a machine learning model using a processor, wherein the raw trial data is patient data. At step 304, plurality of independent features of the raw trial data is identified by the trained machine learning model. At step 306, actionable features from the plurality of independent features are screened by the trained machine learning model. The actionable features show opposite impact between treatment arm patients and control arm patient of the clinical trial. At step 308, cut off range values for each of the actionable features is computed by the processor and a plurality of sub-groups are formed. At step 310, patient response of each of the plurality of sub-groups of patients is simulated by the processor. At step 312, a sub-group of patients is identified from the plurality of sub-groups based upon population percentage and delta response obtained from the simulations that shows optimal clinical trial results in the simulated patient response.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A system for optimizing trial design in a clinical trial, wherein the system includes a computer system comprising a processor communicably coupled to a memory, the processor being configured to:
process and structure raw trial data to a format suitable for input to train a machine learning model, wherein the raw trial data is patient data;
identify plurality of independent features of the raw trial data, wherein the identification of the plurality of independent features is performed using the trained machine learning model;
screen actionable features from the plurality of independent features using the trained machine learning model, wherein actionable features show opposite impact between treatment arm patients and control arm patient of the clinical trial;
compute cut off range values for each of the actionable features, and form a plurality of sub-groups of patients using different combinations of cut-off range values, wherein the cut off range values define an upper limit and a lower limit for values of the actionable features;
simulate patient response of each of the plurality of sub-groups of patients; and
identify a sub-group of patients from the plurality of sub-groups, based upon population percentage and delta response obtained from the simulations, that shows optimal clinical trial results in the simulated patient response.

2. The system of claim 1, wherein the plurality of independent features have missing values in the raw trial data that are imputed using a plurality of imputation techniques, wherein the plurality of imputation techniques employ statistical extrapolation.

3. The system of claim 1, wherein the machine learning model is XGBoost regressor, and wherein the XGBoost regressor is trained using grid search.

4. The system of claim 3, wherein the XGBoost regressor identifies the independent features that do not impact efficacy of treatment used in the clinical trial.

5. The system of claim 1, wherein the plurality of independent features comprise at least one of: genetic features, baseline indexes, vital signs, underlying conditions, medical history, and demographics such as age, gender, height, weight, BMI, nationality, race.

6. The system of claim 1, wherein opposite impact between treatment arm patients and control arm patients is measured as improvement in the treatment arm patients and decrease in efficacy in the control arm patients.

7. A method for optimizing trial design in a clinical trial, wherein the method comprises:
processing and structuring raw trial data to a format suitable for input to train a machine learning model using a processor, wherein the raw trial data is patient data;
identifying plurality of independent features of the raw trial data, wherein the identification of the plurality of independent features is performed using the trained machine learning model;
screening actionable features from the plurality of independent features using the trained machine learning model, wherein actionable features show opposite impact between treatment arm patients and control arm patient of the clinical trial;
computing cut off range values for each of the actionable features, and form a plurality of sub-groups of patients using different combinations of cut-off range values, wherein the cut off range values define an upper limit and a lower limit for values of the actionable features;
simulating patient response of each of the plurality of sub-groups of patients; and
identifying a sub-group of patients from the plurality of sub-groups, based upon population percentage and delta response obtained from the simulations, that shows optimal clinical trial results in the simulated patient response.

8. The method of claim 7, wherein the method comprises imputing the missing values of the plurality of independent features in the trial data using a plurality of imputation techniques, wherein the plurality of imputation techniques employ statistical extrapolation.

9. The method of claim 7, wherein the method comprises training XGBoost regressor using grid search, wherein the machine learning model is XGBoost regressor.

10. The method of claim 9, wherein the method comprises identifying the independent features that do not impact efficacy of treatment used in the clinical trial using the XGBoost regressor.

11. The method of claim 1, wherein the method comprises the plurality of independent features to be at least one of: genetic features, baseline indexes, vital signs, underlying conditions, medical history, and demographics such as age, gender, height, weight, BMI, nationality, race.

12. The method of claim 1, wherein the method comprises measuring opposite impact between treatment arm patients and control arm patients as improvement in the treatment arm patients and decrease in efficacy in the control arm patients.
